(19) United States
(12) Patent Application Publication
(10) Pub. No.: US 2024/0024030 A1
(43) Pub. Date: Jan. 25, 2024

(54) SURGICAL SYSTEM

(71) Applicant: PRECISION AI PTY LTD, Fortitude Valley (AU)

(72) Inventors: Benjamin William Kenny, Fortitude Valley (AU); Sean Michael McMahon, Fortitude Valley (AU)

(21) Appl. No.: 18/260,451

(22) PCT Filed: Aug. 23, 2021
(86) PCT No.: PCT/AU2021/050936

§ 371 Date: Jul. 5, 2023

(30) Foreign Application Priority Data
Jan. 6, 2021 (AU) 2021900016

(51) Int. Cl. A61B 34/10; A61B 90/00

(52) U.S. Cl. CPC A61B 34/10; A61B 90/36; A61B 2034/105; A61B 2034/107; A61B 2034/102; A61B 2034/108; A61B 2034/252

(57) ABSTRACT

A surgical system for use in performing a surgical implant procedure on a biological subject. In a planning phase, a planning processing device acquires scan data indicative of a scan of an anatomical part of the subject and generates model data indicative of an anatomical part model and either a surgical guide model representing a surgical guide, an implant model representing the surgical implant or a tool model representing the surgical tool. A planning visualisation can then be displayed to a user so the user can manipulate the planning visualisation in to calculate a custom guide shape for the surgical guide and/or plan the surgical procedure. During a surgical phase, a surgical guide is used to assist aligning an implant with the anatomical part in use, while a procedure visualisation can be displayed to the user based on the model data.

### BACKGROUND OF THE INVENTION

The present invention relates to a surgical system and method for use in performing a surgical implant procedure on a biological subject, and in one particular example for performing implantation of an orthopaedic prosthesis, such as a shoulder replacement.

### DESCRIPTION OF THE PRIOR ART

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Orthopedic prosthetic implants are used to replace missing joints or bones, or to provide support to a damaged bone, allowing patients receiving implants to regain pain-free motion. Prosthetic implants can be combined with healthy bone to replace diseased or damaged bone, or can replace certain parts of a joint bone entirely. The implants are typically fabricated using stainless steel and titanium alloys for strength, with a coating, such as a plastic coating, being used to acts as an artificial cartilage.

A shoulder replacement is a surgical procedure in which all or part of the glenohumeral joint is replaced by a prosthetic implant, typically to relieve arthritis pain or fix severe physical joint damage. In general, shoulder replacement surgery involves implanting an artificial ball and socket joint including a metal ball that rotates within a polyethylene (plastic) socket. In one approach, the metal ball takes the place of the patient's humeral head and is anchored via a stem, which is inserted down the shaft of the humerus, whilst a plastic socket is placed over the patient's glenoid and secured to the surrounding bone using a cement. However, in reverse shoulder replacement approaches, the ball is attached to the glenoid, whilst the socket is attached to the humerus. In either case, attachment to the humerus typically involves the use of a cutting tool that is attached to the humerus using pins that are drilled into the humeral head, and which is used to cut into the humerus, allowing the implant to be attached.

Irrespective of the approach used, accurate alignment of the ball and socket is important to ensure the replacement joint functions correctly, and any misalignment can cause discomfort and increased joint wear, which in turn can result in the need for additional surgical intervention. Consequently, during the surgical procedure it is important that the ball and socket and accurately aligned when they are attached to the glenoid and humerus.

Whilst guides have been developed to assist with locating the implant on the glenoid, these have varying degrees of success and to date, guides are not available for the humerus. Even where guides are available, the implant process is complex and so careful planning and guidance is desirable to ensure the best outcomes for patients.

WO2020099268 describes a cutting device for the placement of a knee prosthesis comprising a bracket and a cutting guide mounted with the ability to move on said bracket, wherein the bracket comprises a first marker for identifying it and a fixing element for fixing it to a bone, and wherein the cutting guide comprises a second marker for identifying it and a slot defining a cutting plane suited to guiding a cutting tool. The document also relates to an assistance device and to a system comprising said cutting device. The document finally relates to an assistance method and to a computer program product and to a data recording medium for executing the method.

### SUMMARY OF THE PRESENT INVENTION

In one broad form the present invention seeks to provide a surgical system for use in performing a surgical implant procedure on a biological subject, the system including: in a planning phase: a planning display device; one or more planning processing devices configured to: acquire scan data indicative of a scan of an anatomical part of the subject; generate model data indicative of: an anatomical part model generated using the scan data; and, at least one of: a surgical guide model representing a surgical guide used in positioning a surgical implant; an implant model representing the surgical implant; and, a tool model representing the surgical tool used in performing the surgical procedure; cause a planning visualisation to be displayed to a user using the planning display device, the planning visualisation being generated at least in part using the model data; and, manipulate the planning visualisation in accordance with user input commands indicative of interaction with the planning visualisation to at least one of: calculate a custom guide shape for the surgical guide; and, at least partially plan the surgical procedure; and, in a surgical phase: a surgical guide configured to assist in aligning an implant with the anatomical part in use; a procedure display device; and, one or more procedure processing devices configured to cause a procedure visualisation to be displayed to a user using the procedure display device, the procedure visualisation being generated at least in part using the model data and being displayed whilst the surgical procedure is performed.

In one embodiment the one or more planning processing devices use manipulation of the planning visualisation to: determine an operative position of the surgical guide relative to the anatomical part; and, calculate a custom guide shape for the surgical guide based on the operative position.

In one embodiment the one or more planning processing devices are configured to use user input commands to determine an alignment indicative of a desired relative position of the anatomical part model and at least one of: the surgical implant; and, a surgical tool.

In one embodiment the one or more planning processing devices are configured to determine an operative position of the surgical guide relative to the anatomical part at least in part using the alignment.

In one embodiment the one or more planning processing devices are configured to determine the alignment at least in part by having a user at least one of: identify key anatomical features in the representation of the anatomical part model, the alignment being determined based on the key anatomical features; and, position the surgical implant relative to the anatomical part in the visualisation.

In one embodiment the planning visualisation includes one or more input controls allowing a user to adjust the alignment.

In one embodiment the one or more planning processing devices generate procedure data indicative of a sequence of steps representing progression of the surgical implant procedure.

In one embodiment the one or more planning processing devices generate the procedure data at least in part by: causing the planning visualisation to be displayed; using user input commands representing user interaction with the planning visualisation to create each step, each step being indicative of a location and/or movement of at least one of: a surgical tool; a surgical guide; and, a surgical implant; and, generate the procedure data using the created steps.

In one embodiment the one or more procedure processing devices are configured to use the procedure data to cause the procedure visualisation to be displayed.

In one embodiment the one or more procedure processing devices are configured to: determine when a step is complete in accordance with user input commands; and, cause the procedure visualisation to be updated to display a next step.

In one embodiment the procedure visualisation is indicative of at least one of: the scan data; the anatomical part model; a model implant; and, one or more steps.

In one embodiment the one or more procedure processing devices are configured to: determine a procedure display device location with respect to: the surgical guide; or the anatomical part of the subject; and, cause the procedure visualisation to be displayed in accordance with the procedure display device location so that: a visualisation of the surgical guide model is displayed overlaid on the surgical guide; or a visualisation of the anatomical part model is displayed overlaid on the anatomical part of the subject.

In one embodiment the one or more procedure processing devices are configured to determine the procedure display device location by at least one of: using signals from one or more sensors; using user input commands; performing image recognition on captured images; and, detecting coded data present on at least one of the surgical guide, surgical tools and the subject.

In one embodiment the captured images are captured using an imaging device associated with the procedure display device.

In one embodiment the planning or procedure visualisation includes a digital reality visualisation, and wherein the one or more processing devices are configured to allow a user to manipulate visualisation by interacting with at least one of: the anatomical part; the surgical implant; a surgical tool; and, to surgical guide.

In one embodiment at least one of the planning and procedure display devices is at least one of: an augmented reality display device; and, a wearable display device.

In one embodiment the surgical implant includes at least one of: a prosthesis; an orthopaedic shoulder prosthesis; a ball and socket joint; a humeral implant attached to a humeral head of the subject; a glenoidal implant attached to a glenoid of the subject; ball attached via a stem to the humeral head or glenoid of the subject; and, a socket attached using a binding material to the glenoid or humeral head of the subject.

In one embodiment the surgical guide includes a glenoidal guide for attachment to a glenoid of the subject, and wherein the glenoidal guide includes: a glenoidal guide body configured to abut the glenoid in use, the glenoidal guide body including one or more holes for use in guiding attachment of an implant to the glenoid; and, a number of glenoidal guide arms configured to engage an outer edge of the glenoid to secure the glenoidal guide in an operative position.

In one embodiment an underside of the glenoid body is shaped to conform to a profile of the glenoid.

In one embodiment the one or more holes include: a central hole configured to receive a K-wire for guiding positioning of the implant; a superior hole for configured to receive a temporary K-wire used to act as an indicator of rotation and placement of the glenoid implant during insertion; an anterior hole configured to receive a surgical tool used to aid in placement and stability of the guide.

In one embodiment the glenoidal guide arms include: an anterosuperior arm configured to sit and articulate inferior to the coracoid process, and extend across the glenoid vault and over the bony rim of the glenoid in use; an anteroinferior arm configured to sit along the anteroinferior aspect of the glenoid and glenoid vault and extend over the bony rim of the glenoid; and, a posterosuperior arm configured to sit on the bony glenoid rim.

In one embodiment the surgical guide includes a humeral guide for attachment to a humerus of the subject, and wherein the humeral guide includes: a humeral guide body configured to extend from an articular surface of a humeral head down the bicipital groove of the humerus; and, a humeral guide arm configured to extend from the body and including one or more holes configured to receive surgical pins to allow for attachment of a cutting block to the humerus.

In one embodiment an underside of the humeral guide body is shaped to conform to a profile of the humeral head.

In one broad form the present invention seeks to provide a method for performing a surgical implant procedure on a biological subject, the method including: in a planning phase using one or more planning processing devices to: acquire scan data indicative of a scan of an anatomical part of the subject; generate model data indicative of: an anatomical part model generated using the scan data; and, at least one of: a surgical guide model representing a surgical guide used in positioning a surgical implant; an implant model representing the surgical implant; and, a tool model representing the surgical tool used in performing the surgical procedure; cause a planning visualisation to be displayed to a user using the planning display device, the planning visualisation being generated at least in part using the model data; and, manipulate the planning visualisation in accordance with user input commands indicative of interaction with the planning visualisation to at least one of: calculate a custom guide shape for the surgical guide; and, at least partially plan the surgical procedure; and, in a surgical phase: using a surgical guide to assist in aligning an implant with the anatomical part in use; and, using one or more procedure processing devices to display a procedure visualisation to a user using a procedure display device, the procedure visualisation being generated at least in part using the model data and being displayed whilst the surgical procedure is performed.

In one broad form the present invention seeks to provide a surgical system for planning a surgical implant procedure on a biological subject, the system including: a planning display device; one or more planning processing devices configured to: acquire scan data indicative of a scan of an anatomical part of the subject; generate model data indicative of: an anatomical part model generated using the scan data; and, at least one of: a surgical guide model representing a surgical guide used in positioning a surgical implant; an implant model representing the surgical implant; and, a tool model representing the surgical tool used in performing the surgical procedure; cause a planning visualisation to be displayed to a user using the planning display device, the planning visualisation being generated at least in part using the model data; and, manipulate the planning visualisation in accordance with user input commands indicative of interaction with the planning visualisation to at least one of: calculate a custom guide shape for the surgical guide; and, at least partially plan the surgical procedure.

In one broad form the present invention seeks to provide a surgical system for performing a surgical implant procedure on a biological subject, the system including: a surgical guide configured to assist in aligning an implant with the anatomical part in use; a procedure display device; and, one or more procedure processing devices configured to cause a procedure visualisation to be displayed to a user using the procedure display device, the procedure visualisation being generated at least in part using model data and being displayed whilst the surgical procedure is performed.

In one broad form the present invention seeks to provide a method for planning a surgical implant procedure on a biological subject, the method including using one or more planning processing devices to: acquire scan data indicative of a scan of an anatomical part of the subject; generate model data indicative of: an anatomical part model generated using the scan data; and, at least one of: a surgical guide model representing a surgical guide used in positioning a surgical implant; an implant model representing the surgical implant; and, a tool model representing the surgical tool used in performing the surgical procedure; cause a planning visualisation to be displayed to a user using the planning display device, the planning visualisation being generated at least in part using the model data; and, manipulate the planning visualisation in accordance with user input commands indicative of interaction with the planning visualisation to at least one of: calculate a custom guide shape for the surgical guide; and, at least partially plan the surgical procedure.

In one broad form the present invention seeks to provide a method for performing a surgical implant procedure on a biological subject, the method including: using a surgical guide generated using a to assist in aligning an implant with the anatomical part in use; and, using one or more procedure processing devices to display a procedure visualisation to a user using a procedure display device, the procedure visualisation being generated at least in part using model data and being displayed whilst the surgical procedure is performed.

In one broad form the present invention seeks to provide a humeral guide for a shoulder prosthesis implant procedure, the humeral guide being for attachment to a humerus of the subject, and including: a humeral guide body configured to extend from an articular surface of a humeral head down the bicipital groove of the humerous; and, a humeral guide arm configured to extend from the body and including one or more holes configured to receive surgical pins to allow for attachment of a cutting block to the humerous.

In one embodiment an underside of the humeral guide body is shaped to conform to a profile of the humeral head.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which: —

FIG. 1 is a flow chart of an example of a method for use in performing a surgical implant procedure on a biological subject;

FIG. 2 is a schematic diagram of a distributed computer architecture;

FIG. 3 is as schematic diagram of an example of a processing system;

FIG. 4 is a schematic diagram of an example of a client device;

FIG. 5 is a schematic diagram of an example of a display device;

FIGS. 6A and 6B are a flow chart of an example of a method for use in manufacturing a custom guide during a pre-surgical planning phase;

FIGS. 7A to 7F are screen shots showing a first example of a user interface used during the pre-surgical planning phase;

FIGS. 7G and 7H are screen shots showing a second example of a user interface used during the pre-surgical planning phase;

FIGS. 8A to 8C are schematic diagrams of an example of a glenoid guide;

FIGS. 8D to 8F are schematic diagrams of the glenoid guide of FIGS. 8A to 8C attached to a glenoid;

FIGS. 9A to 9C are schematic diagrams of an example of a humeral guide;

FIGS. 9D to 9F are schematic diagrams of the humeral guide of FIGS. 9A to 9C attached to a humerus;

FIG. 10 is a flow chart of an example of a method for use in planning a procedure during a pre-surgical planning phase;

FIG. 11 is a flow chart of an example of a method for use in performing a procedure during a surgical phase;

FIGS. 12A to 12C are screen shots showing an example of a user interface used during the surgical phase;

FIG. 13 is a flow chart of an example of a method for use in aligning a procedure visualisation with a subject; and,

FIGS. 14A and 14B are graphs illustrating results of a study of the accuracy of placement of implants using the surgical guides generated using the system and method.

### DETAILED DESCRIPTION OF THE PREFERRED EMBODIMENTS

An example of a system and method for use in performing a surgical implant procedure on a biological subject will now be described.

For the purpose of illustration, it is assumed that the process involves a pre-surgical planning phase, and a surgical phase, in which a surgical implant is implanted into a subject.

During the planning phase, the process is performed at least in part using one or more planning electronic processing devices and one or more planning displays, which optionally form part of one or more processing systems, such as computer systems, or the like, optionally including a separate display device, such as a digital reality headset. The planning processing devices are used to generate models and visualisations that can assist in planning the surgical implant procedure, and in one example, are used to create a custom shape for a surgical guide used in the procedure.

The surgical guide is manufactured and used during the surgical phase to guide positioning of a surgical implant and/or one or more surgical tools. Additionally, during the surgical phase, the system uses one or more procedure electronic processing devices and one or more procedure displays, which again optionally form part of one or more processing systems, such as computer systems, servers, or the like, with the display device optionally being a separate device, such as a digital reality headset, or the like. The procedure processing devices and displays are used to display visualisations that can assist a surgeon in performing the surgical implant procedure, for example, to show the surgeon where guides, implants or surgical tools should be located relative to a subject's anatomy.

Whilst reference is made to separate planning and procedure processing devices and planning and procedure displays, this is largely to distinguish between devices used in the different phases, but it will be appreciated that in practice these could be the same physical devices. In other words, the same processing devices and/or displays could be used in both planning and surgical phases, although different devices could be used depending on the preferred implementation.

The system can use multiple processing devices, with processing performed by one or more of the devices. However, this is not essential and a single planning and/or procedure processing device could be used. Accordingly, for ease of illustration, the following examples will refer to a single device, but it will be appreciated that reference to a singular processing device should be understood to encompass multiple processing devices and vice versa, with processing being distributed between the devices as appropriate.

The terms “biological subject”, “subject,” “individual” and “patient” are used interchangeably herein to refer to an animal subject, particularly a vertebrate subject, and even more particularly a mammalian subject, such as a human. Suitable vertebrate animals that fall within the scope of the invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards, etc.), and fish. A preferred subject is a primate (e.g., a human, ape, monkey, chimpanzee).

The term “user” is intended to refer to an individual using the surgical system and/or performing the surgical method. The individual is typically medically trained and could include a clinician and/or surgeon depending on the procedure being performed. Although reference is made to a single user, it will be appreciated that this should be understood to encompass multiple users, including potentially different users during planning and procedure phases, and reference to a single user is not intended to be limiting

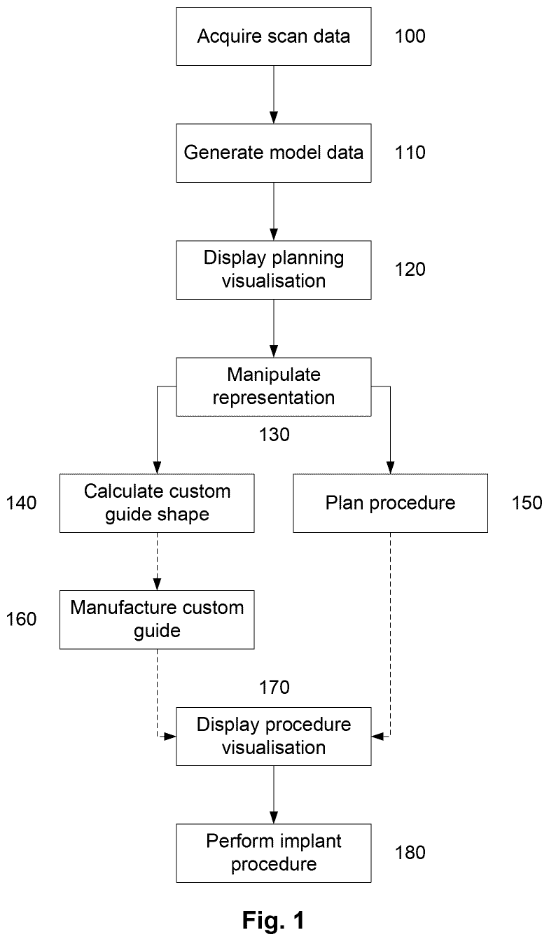

An example of operation of the surgical system will now be described with reference to FIG. 1.

In this example, at step 100, the planning processing device acquires scan data indicative of a scan of an anatomical part of the subject. The scan data can be of any appropriate form, and this may depend on the nature of the implant and the procedure being performed. For example, in the case of a shoulder reconstruction, the scan data would typically include CT (Computerized Tomography) scan data, whereas other procedures may MRI (Magnetic Resonance Imaging) scans, or the like. The scan data can be acquired in any appropriate manner, but this typically involves retrieving the scan data from a database or similar, although scan data could be received directly from a scanner.

At step 110, the planning processing device generates model data indicative of at least an anatomical part model generated using the scan data. The anatomical part will vary depending on the procedure being performed, but in the case of an orthopaedic implant, the anatomical part will typically include one or more bones. Thus, for example, in the case of a shoulder replacement, the anatomical part model will typically include models of a subject's humerus and scapula. The model data is typically in the form of a CAD (Computer Aided Design) model, and can be generated using known techniques. For example, scans can be analysed to detect features in the scans, such as edges of bones, with the model data being generated by using multiple scan slices to reconstruct the shape of the respective bone, and hence generated model data.

Model data is also generated for a surgical guide model representing a surgical guide used in positioning a surgical implant. This is typically based on a template indicative of an approximate shape for the resulting guide. The model data may also include models of surgical implants and/or surgical tools used in performing the implant. It will be appreciated that the surgical implant and surgical tools are typically standard implants and tools, and so model data for each of these components can be derived from manufacturer specifications for the implants and/or tools, and could for example be predefined and retrieved from a database, or similar, as required. This allows models of the surgical tool and/or implant to be readily incorporated into a model for a given procedure, in turn allowing alignments to be calculated and visualisations to be generated as needed.

At step 120, the planning processing device causes a planning visualisation to be displayed to a user using the planning display device. The user is typically a clinician, such as a surgeon, that is to be involved in performing the procedure, although this is not essential and the other user could include any appropriate person that is capable of using the system to assist in preparing for the surgical procedure to be performed. The planning visualisation is generated based on the model data, and could for example include a visual representation of the anatomical part of the subject, as well as the surgical guide and/or one or more of the surgical implant or surgical tool used in performing the procedure. The visualisation could be presented on a display screen, for example in the form of a two-dimensional image. Additionally, and/or alternatively, the visualisation could be presented in the form of a digital reality visualisation, such as an augmented, mixed and/or virtual reality visualisation, displayed using an appropriate display device such as a VR or AR headset or similar.

The visualisation is used to assist the user in visualising the surgical procedure, with interaction with user input commands indicative of interaction with the planning visualisation being used to allow the user to manipulate model components, for example to visualise different implant, tool or guide positions relative to the anatomical parts. As part of this process, at step 130 the planning processing device uses the user input commands to manipulate the visualisation, for example to have the user move model parts relative to each other.

This can be performed in order to calculate a custom guide shape at step 140. For example, this can be used to determine an operative position of the surgical guide relative to the anatomical part, with this being used to ascertain the custom shape of the guide so that the guide when attached to the anatomical part of the subject will sit in the operative position. This process can be achieved either by having the user define a desired position of the surgical guide relative to the anatomical part, or by having the user define a desired alignment of the surgical tool or implant relative to the anatomical part, with the operative position of the surgical guide being calculated based on the alignment.

The custom shape is typically derived at least in part from a default shape for the surgical guide, such as a template shape, with modifications to the default shape being performed to customise the surgical guide for the subject, based on the shape of the relevant subject anatomy. For example, in the case of a glenoidal guide, the shape of the guide can be modified so that it conforms to the actual shape of the subject's glenoid. This ensures that the surgical guide attaches to the subject anatomy in a unique position and orientation, and hence correctly aligns with the relevant subject anatomy.

Additionally, and/or alternatively, manipulation of the visualisation can be used to help plan the surgical procedure at step 150. For example, this could be used as ascertain a desired position, alignment and/or movement of the surgical implant, tools or guide, that would be required in order to complete the surgical procedure.

In either case, this can be a wholly manual process, for example allowing the user manually define the operative position and/or alignment, or could be an automated or semi-automated process. For example, key markers could be identified on the anatomical part, with the processing device then calculating an optimum operative position and/or alignment based on the markers, with the user then optionally refining this as needed.

In the event that a custom guide shape has been calculated, this can be used to manufacture the guide at step 160, for example using additive or subtractive manufacturing techniques, such as 3D printing, or the like, with the exact technique used depending on the nature of the guide and the preferred implementation. It will be appreciated that the manufacturing step can be performed in any appropriate manner, but this typically involves generating an STL (Standard Tessellation Language) file based on the custom shape, and then making the file available for use by a 3D printer or similar. The surgical guides are typically manufactured using a resilient bio-compatible polymer or resin, such as NextDent SG™, or the like.

Example guides for a shoulder replacement, including a glenoidal guide and a humeral guide, will be described in more detail below.

At step 170, the procedure processing device is used to display a procedure visualisation, which is generated based on the model data and is displayed whilst the surgical procedure is performed. This can be used to assist a user, such as a surgeon, in performing the surgical implant procedure at step 180.

In one example, this is achieved by displaying one or more steps of the implant procedure, for example, displaying a visualisation of the surgical guide in an operative position, so that the surgeon can confirm that they have correctly positioned the guide. Again the procedure visualisation could be of any form, but in one example, is displayed as a digital reality, and in particular, augmented reality, visualisation. This approach allows the visualisation to be displayed via a headset, or glasses arrangement, such as Hololens™, or similar, allowing the user to view the visualisation concurrently with the actual surgical situation, so the user can perform the surgical procedure whilst simultaneously viewing the procedure visualisation. This allows the user to more easily perform a visual comparison and assess that the procedure is being performed as planned, as well as providing the user with access to pertinent information, such as patient details or similar, which can assist in ensuring the procedure is performed appropriately.

Accordingly, the above described arrangement provides a system and process for assisting with a surgical procedure. The system operates in two phases, namely a planning phase, during which a custom guide is created and/or plan is created, and a subsequent surgical phase, in which the custom guide and/or plan is used in performing the surgical procedure. However, whilst reference is made to distinct phases, it will be appreciated that these could be performed partially concurrently, depending on the implementation. For example, as will be described in more detail below, the planning phase can be used to plan steps performed in the procedure. In this example, if difficulties arise in a surgical procedure, one or more clinicians external to an operating theatre may perform additional planning to allow assist a surgeon performing the procedure. Accordingly, whilst the planning phase is typically performed prior to the surgical phase, this is not intended to be limiting.

The system creates a surgical guide and/or plan in the planning phase by displaying visualisations including a representation of the subject's anatomical part, such as the shoulder glenoid or humerus, together with an implant, surgical tool or guide, allowing the user to manipulate these components, for example to define a desired implant or tool alignment and/or an optimum operative position for the surgical guide. This information is then used with a 3D model of the user's anatomy to generate a custom guide shape, so that the guide is customised for the subject, and can only attach to the subject in a correct orientation and/or to create a surgical plan.

Following this, in a procedure phase, visualisations can be used to further assist the user in ensuring the surgical procedure is being performed correctly, and specifically that the implant, tools and guides are provided in a correct alignment and/or operative position. These processes, when used in conjunction, help ensure implants are implanted correctly, and this in turn reduces adverse outcomes for subjects.

A number of further features will now be described.

As previously mentioned, the planning visualisation could be indicative of the anatomical part and the surgical guide, allowing the user to manipulate the visualisation to define an operative position for the guide. In practice, however, the operative position of the guide is less important than alignment of the implant and/or surgical tool, and so accordingly, more typically a planning visualisation is generated that is indicative of the anatomical part and the surgical implant or surgical tool. The user then interacts with the visualisation, optionally though a combination or manually and/or automated processes, allowing an alignment to be determined which is indicative of desired relative position of the anatomical part model and either the surgical implant or the surgical tool. This can then be used to calculate an operative position for the surgical guide that should be used in order for the alignment to be realised. It will be appreciated that alignment of the surgical implant and/or surgical tool can additionally and/or alternatively be used in performing planning, for example, to allow a visualisation of a desired surgical implant position to be created for visual inspection by a surgeon during the surgical procedure.

In practice, the process of determining the alignment could include having the identify key anatomical features in the representation of the anatomical part model, with the alignment being determined based on the key anatomical features and/or position the surgical implant relative to the anatomical part in the visualisation. For example, key features, such as a centre of the glenoid, the trigonum and inferior angle of the scapula could be marked manually, with this being used to automatically calculate positioning of transverse and scapula planes, which are then used together with the centre of the glenoid to propose an initial alignment. This can then be refined manually through manipulation of the visualisation, until the user is happy with the resulting alignment.

Adjustment of the alignment could be achieved using any suitable technique, and could include the use of an input device, such as a mouse and/or keyboard. However, particularly when a digital reality visualisation is used, this could include one or more input controls, such as sliders or the like, to be presented as part of the visualisation, allowing a user to adjust the alignment as needed.

In one example, the planning phase can involve having the planning processing device generate procedure data indicative of a sequence of steps representing progression of the surgical implant procedure. For example, this could involve defining each of the key steps involved in the procedure, such as positioning of the guide, reaming the bone, attachment of securing pins, cutting, and alignment and attachment of the implant. These can serve as a useful guide to the user when they are performing the procedure in practice.

The procedure data are typically generated at least in part by causing the planning visualisation to be displayed including the anatomical part, and the implant, surgical guide and/or surgical instrument(s), as appropriate to the relevant step of the procedure. User input commands are then used to allow the user to interact with and manipulate the planning visualisation, for example to define a desired location and/or movement of the implant, surgical guide and/or surgical instrument(s), needed to implement the relevant step. Once this has been performed, procedure data indicative of the desired location/movement can be generated, allowing visualisations of the steps to be recreated during the surgical phase.

This, in turn, allows the procedure processing device to use the procedure data to cause the procedure visualisation to be displayed. Thus, the procedure visualisation can include visualisations of the one or more steps of the procedure, with each step showing a representation of the anatomical part of the subject, and the desired relative positioning of the surgical implant, surgical guide or surgical tool. In one particular example, the procedure processing device is configured to determine when a step in the procedure is completed, for example based on user input commands, and then update the procedure visualisation so that the visualisation displays a next step. Thus, it will be appreciated that in practice, when performing the procedure, the user can be presented with a visualisation of a step. The user confirms with a suitable input command, when the step is complete, causing a next step to be displayed. This allows the user to simply follow the pre-defined steps in turn, and thereby effectively carry out the surgical procedure.

To further enhance use of the procedure visualisation when using an augmented reality display, the procedure processing device can be configured to determine a procedure display device location with respect to the surgical guide and or anatomical part of the subject, and then cause the procedure visualisation to be displayed in accordance with the procedure display device location. This can be done so that the visualisation of the surgical guide model is displayed overlaid on the real physical surgical guide and/or a visualisation of the anatomical part model is displayed overlaid on the anatomical part of the subject, which can help the user ensure components are correctly aligned in practice.

To determine the procedure display device location, the procedure processing device can use a variety of different techniques, depending on the preferred implementation. For example, this could use signals from one or more sensors to localise the procedure display device and the subject in an environment, such as an operating theatre, using the localisation to determine the relative position. Alternatively, this could be achieved using user input commands, for example, by displaying a visualisation of the subject anatomy statically within a field of view of the display device, moving the display device until the visualisation is aligned with the subject anatomy, and then using user input commands to confirm the alignment. A similar approach could be achieved by performing image recognition on captured images, and in particular, images captured using an imaging device forming part of the display device. In a further example, this could be achieved by detecting coded data, including fiducial markings, such as QR codes, April Tags, or infrared navigation markers present on the surgical guide, surgical guide and/or patient anatomy. In this example, analysis of the markings can be used to ascertain the relative position of the display device and the subject anatomy or surgical guide.

As previously mentioned, the planning and/or procedure visualisation can include a digital reality visualisation, such as virtual or augmented reality visualisation. Such visualisations are particularly beneficial as these allow a user to view representations of the surgical procedure in three dimensions, enabling the user to manipulate one or more of the anatomical part, the surgical implant, the surgical tool and/or surgical guide, thereby ensuring these are correctly positioned, both in the planning visualisation and in the actual surgical procedure. In this case, the display devices can be augmented reality display devices and optionally wearable display devices, such as augmented reality glasses, goggles, or headsets, although it will be appreciated that other suitable display devices could be used. For example, a tablet or other similar display device could be provided within an operating theatre, so that this can be moved into position to capture images of the surgical procedure, with the visualisations being displayed overlaid on the captured images, to thereby provide a mixed reality visualisation.

It will be appreciated that the above described process and system could be used in a wide range of implant situations and could be used for example when the surgical implant includes any prosthesis. In one particular example, this can be used when the prosthesis is an orthopaedic shoulder prosthesis, in which case the prosthesis typically includes a ball and socket joint, including a humeral implant attached to a humeral head of the subject and a glenoidal implant attached to a glenoid of the subject. In this example, the prosthesis could include a ball attached via a stem to the humeral head or glenoid of the subject and a socket attached using a binding material to the glenoid or humeral head of the subject.

When the prosthesis is an orthopaedic shoulder prosthesis, the surgical guide typically includes a glenoid guide for attachment to a glenoid of the subject, and a humeral guide for attachment to a humerus of the subject.

The glenoid guide typically includes a glenoid guide body configured to abut the glenoid in use, the glenoid guide body including one or more holes for use in guiding attachment of an implant to the glenoid and a number of glenoid guide arms configured to engage an outer edge of the glenoid to secure the glenoid guide in an operative position. In this regard, the arms are configured to secure the glenoid guide body to the glenoid, so that an underside of the glenoid body abuts against the glenoid. The arms typically include an anterosuperior arm configured to sit and articulate inferior to the coracoid process, and extend across the glenoid vault and over the bony rim of the glenoid in use, an anteroinferior arm configured to sit along the anteroinferior aspect of the glenoid and glenoid vault and extend over the bony rim of the glenoid and a posterosuperior arm configured to sit on the bony glenoid rim.

In this example, an underside of the glenoid body is shaped to conform to a profile of the glenoid, and this in conjunction with the configuration of the arms, ensures the glenoid guide can only be attached to the glenoid in a particular orientation, position and alignment, which in turn ensures the holes are at defined positions relative to the glenoid.

In one example, the holes include a central hole configured to receive a K-wire for guiding positioning of the implant, a superior hole for configured to receive a temporary K-wire used to act as an indicator of rotation and placement of the glenoid implant during insertion, and an anterior hole configured to receive a surgical tool used to aid in placement and stability of the guide. However, it will be appreciated that other holes arrangements could be used depending on the preferred implementation.

By contrast, the humeral guide typically includes a humeral guide body configured to extend from an articular surface of a humeral head down the bicipital groove of the humerus and a humeral guide arm configured to extend from the body and including one or more holes configured to receive surgical pins to allow for attachment of a cutting block to the humerus. In this example, an underside of the humeral guide body is shaped to conform to a profile of the humeral head.

Thus, this arrangement uses the shape of the humeral head to locate the humeral guide, so that the body is at a fixed position and orientation relative to the humeral head. Holes in the humeral head are created by drilling and/or reaming the bone, allowing the surgical pins to be inserted into the bone, at which point the guide can be removed. With the pins in place, these act to locate the cutting tool, so that the humeral head can be cut in a desired location so as to receive the implant.

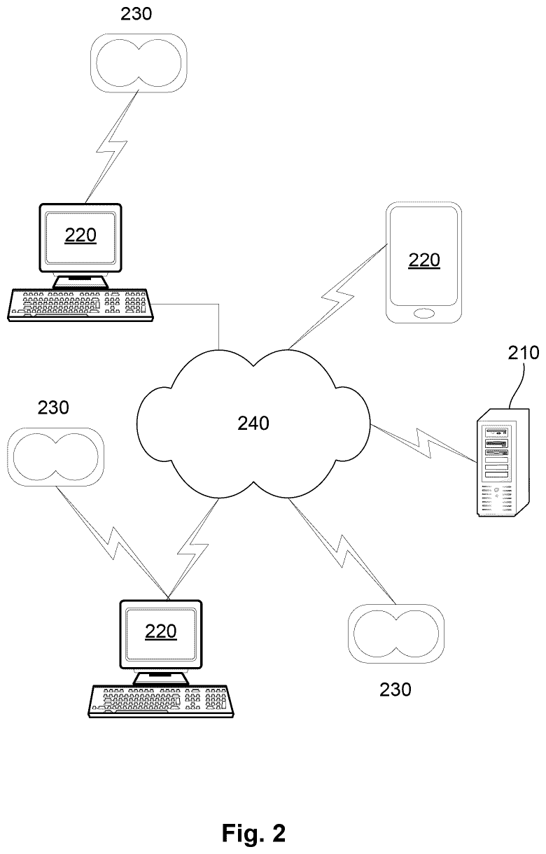

An example of a system for performing the above described surgical procedure will now be described in more detail with reference to FIGS. 2 to 5.

In this example, the system includes a processing system 210, such as one or more servers, provided in communication with one or more client devices 220, via one or more communications networks 240. One or more display devices 230 can be provided, which are optionally in communication with the client devices 220, and/or the processing system 210, via the network 240. It will be appreciated that the configuration of the networks 240 are for the purpose of example only, and in practice the processing system 210, client devices 220, and display devices 230 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

Whilst the processing system 210 is shown as a single entity, it will be appreciated that in practice the processing system 210 can be distributed over a number of geographically separate locations, for example as part of a cloud-based environment. However, the above described arrangement is not essential and other suitable configurations could be used.

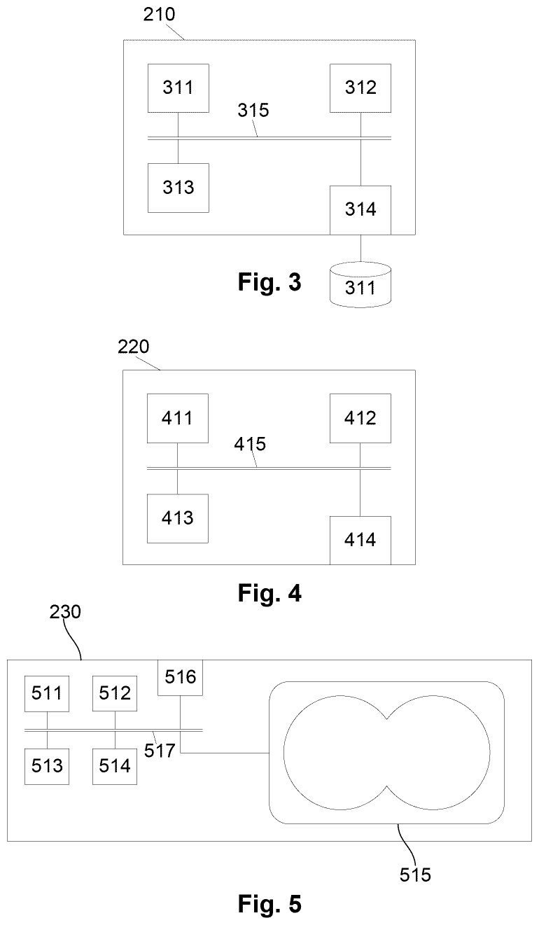

An example of a suitable processing system 210 is shown in FIG. 3. In this example, the processing system 210 includes at least one microprocessor 311, a memory 312, an optional input/output device 313, such as a keyboard and/or display, and an external interface 314, interconnected via a bus 315 as shown. In this example the external interface 305 can be utilised for connecting the processing system 210 to peripheral devices, such as the communications networks 240, databases, other storage devices, or the like. Although a single external interface 315 is shown, this is for the purpose of example only, and in practice multiple interfaces using various methods (e.g. Ethernet, serial, USB, wireless or the like) may be provided.

In use, the microprocessor 311 executes instructions in the form of applications software stored in the memory 312 to allow the required processes to be performed. The applications software may include one or more software modules, and may be executed in a suitable execution environment, such as an operating system environment, or the like.

Accordingly, it will be appreciated that the processing system 210 may be formed from any suitable processing system, such as a suitably programmed client device, PC, web server, network server, or the like. In one particular example, the processing system 210 is a server, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

As shown in FIG. 4, in one example, the client device 220 includes at least one microprocessor 411, a memory 412, an input/output device 413, such as a keyboard and/or display, and an external interface 414, interconnected via a bus 415 as shown. In this example the external interface 414 can be utilised for connecting the client device 220 to peripheral devices, such as a display device 230, the communications networks 240, databases, other storage devices, or the like. Although a single external interface 414 is shown, this is for the purpose of example only, and in practice multiple interfaces using various methods (e.g. Ethernet, serial, USB, wireless or the like) may be provided.

In use, the microprocessor 411 executes instructions in the form of applications software stored in the memory 412 to allow for communication with the processing system 210 and/or display device 230, as well as to allow user interaction for example through a suitable user interface.

Accordingly, it will be appreciated that the client devices 220 may be formed from any suitable processing system, such as a suitably programmed PC, Internet terminal, lap-top, or hand-held PC, a tablet, or smart phone, or the like. Thus, in one example, the client device 220 is a standard processing system such, which executes software applications stored on non-volatile (e.g., hard disk) storage, although this is not essential. However, it will also be understood that the client devices 220 can be any electronic processing device such as a microprocessor, microchip processor, logic gate configuration, firmware optionally associated with implementing logic such as an FPGA (Field Programmable Gate Array), or any other electronic device, system or arrangement.

The display device 230 includes at least one microprocessor 511, a memory 512, an optional input/output device 513, such as a keypad or input buttons, one or more sensors 514, a display 515, and an external interface 516, interconnected via a bus 517 as shown in FIG. 5.

The display device 230 can be in the form of HMD (Head Mounted Display), and is therefore provided in an appropriate housing, allowing this to be worn by the user, and including associated lenses, allowing the display to be viewed, as will be appreciated by persons skilled in the art.

In this example, the external interface 516 is adapted for normally connecting the display device to the processing system 310 or client device 320 via a wired or wireless connection. Although a single external interface 516 is shown, this is for the purpose of example only, and in practice multiple interfaces using various methods (eg. Ethernet, serial, USB, wireless or the like) may be provided. In this particular example, the external interface would typically include at least a data connection, such as USB, and video connection, such as DisplayPort, HMDI, Thunderbolt, or the like.

In use, the microprocessor 511 executes instructions in the form of applications software stored in the memory 512 to allow the required processes to be performed. The applications software may include one or more software modules, and may be executed in a suitable execution environment, such as an operating system environment, or the like. Accordingly, it will be appreciated that the processing device could be any electronic processing device such as a microprocessor, microchip processor, logic gate configuration, firmware optionally associated with implementing logic such as an FPGA (Field Programmable Gate Array), a Graphics Processing Unit (GPU), an Application-Specific Integrated Circuit (ASIC), a system on a chip (SoC), digitial signal processor (DSP), or any other electronic device, system or arrangement.

The sensors 514 are generally used for sensing an orientation and/or position of the display device 230, and could include inertial sensors, accelerometers or the like. Additional sensors, such as light or proximity sensors could be provided to determine whether the display device is currently being worn, whilst eye tracking sensors could be used to provide an indication of a point of gaze of a user. This information is generally provided to the processing system 210 and/or client device 220, allowing the position and/or orientation of the display device 230 to be measured, in turn allowing images generated by the processing system 210 and/or client device 220 to be based on the display device position and/or orientation, as will be appreciated by persons skilled in the art.

For the purpose of the following examples, it is assumed that one or more processing systems 210 are servers, which communicate with the client devices 220 via a communications network, or the like, depending on the particular network infrastructure available. The servers 210 typically execute applications software for performing required tasks including storing and accessing data, and optionally generating models and/or visualisations, with actions performed by the servers 210 being performed by the processor 311 in accordance with instructions stored as applications software in the memory 312 and/or input commands received from a user via the I/O device 313, or commands received from the client device 220.

It will also be assumed that the user interacts with the client device 220 via a GUI (Graphical User Interface), or the like presented on a display of the client device 220, and optionally the display device 230. Where a separate display device 230 is used, the client device 220 will also typically receive signals from the display device 230, and use these to determine user inputs and/or a display device position and/or orientation, using this information to generate visualisations, which can then be displayed using the display device 230, based on the position and/or orientation of the display device 230. Actions performed by the client devices 220 are performed by the processor 411 in accordance with instructions stored as applications software in the memory 412 and/or input commands received from a user via the I/O device 502.

However, it will be appreciated that the above described configuration assumed for the purpose of the following examples is not essential, and numerous other configurations may be used. It will also be appreciated that the partitioning of functionality between the client devices 220, and the servers 210 may vary, depending on the particular implementation.

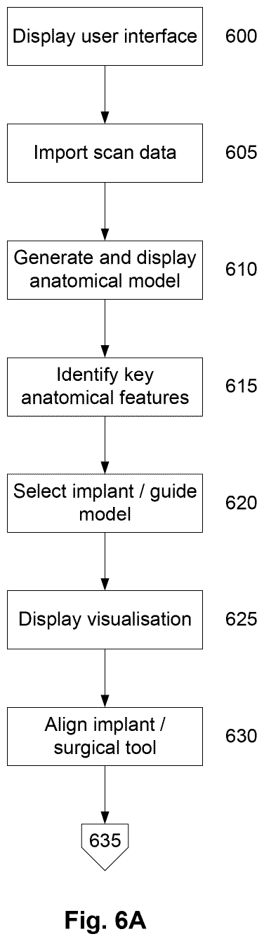

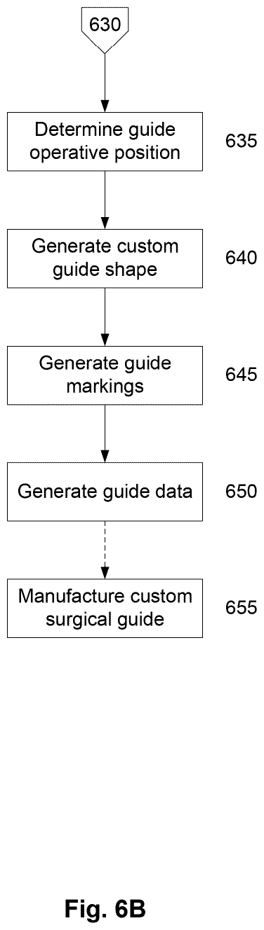

An example of the process for designing a custom surgical guide will now be described with reference to FIGS. 6A and 6B.

In this example, the client device 220 displays a user interface at step 600. The user interface can be displayed on a display of the client device and/or on a separate display device 230, depending on a user preference and/or the preferred implementation. At step 605, the user selects scan data to import, typically based on an identity of a subject on which the surgical procedure is being performed, with this being used to generate an anatomical model at step 610. This process can be performed locally by the client device 220, but as this can be computationally expensive, and so may be performed by the server 210, with the model being uploaded to the client device 220 for display and use.

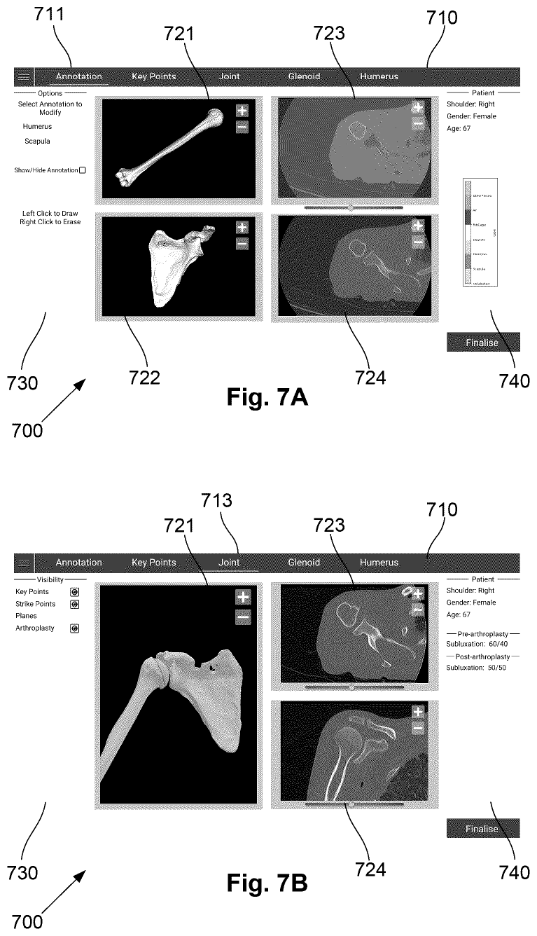

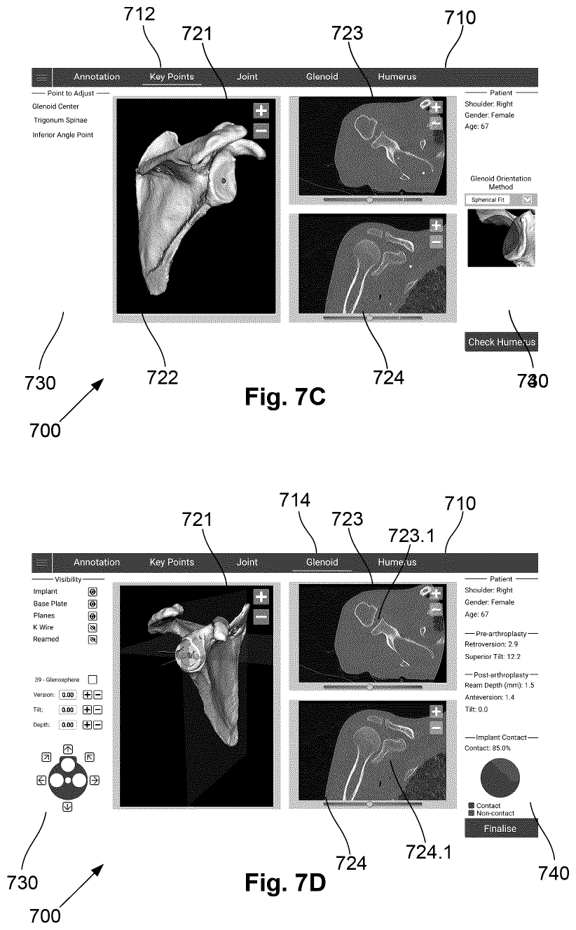

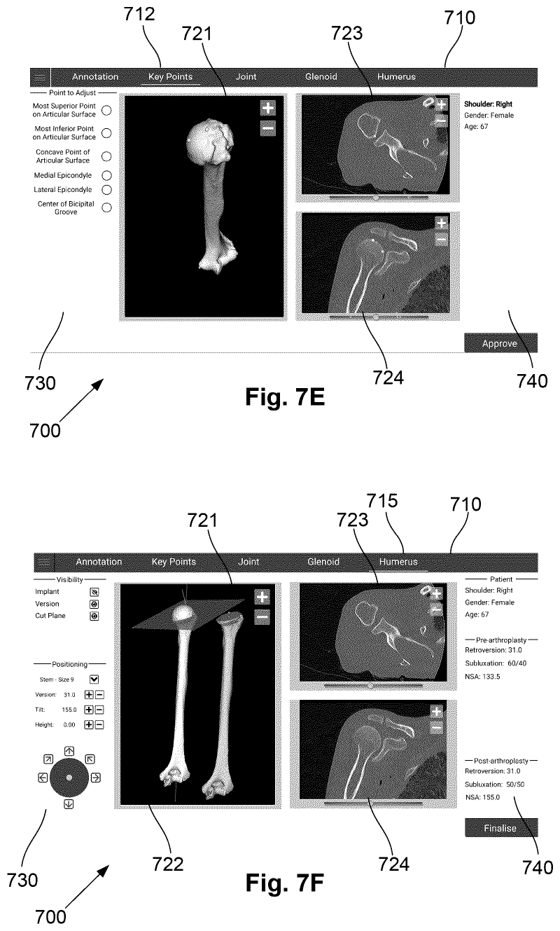

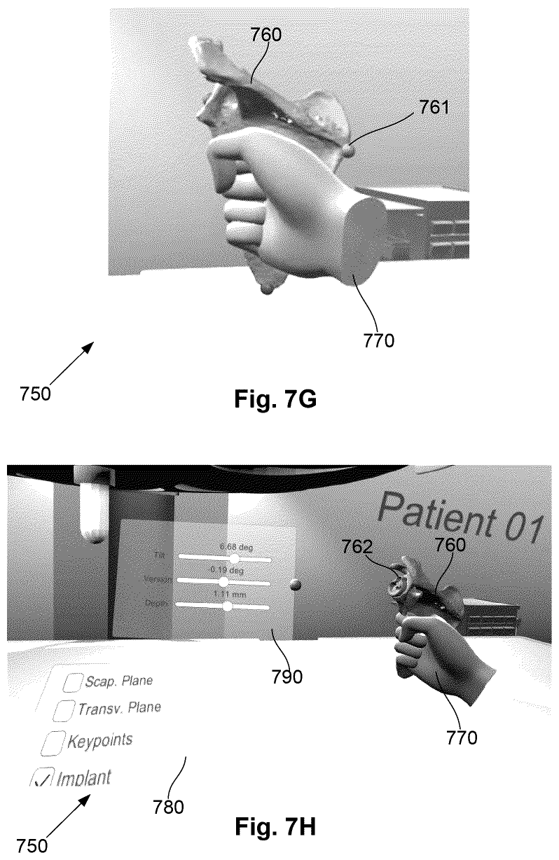

Once generated, the anatomical model can then be displayed as part of the user interface and examples of this are shown in FIGS. 7A to 7H.

In the example of FIG. 7A, the user interface 700 includes a menu bar 710, including a number of tabs allowing a user to select different information to view. In this example an annotation tab 711 is selected allowing a user to annotate information. The user interface further incudes windows 721, 722, 723, 724. In this example, the windows 723, 724 show scan data, measured for the subject, whilst the windows 721, 722 show 3D models of the humerus and scapula that have been generated from the scan data. A left side bar 730 provides one or more input controls, whilst the right side bar 740 displays information, with the content of the side bars 730, 740 varying depending on the tab selected in the menu bar 710. In this instance input controls are provided in the left side bar 730 to allow annotation of the models and/or scan data, whilst patient information is displayed in the right side bar 740.

In the example of FIG. 7B, a joint tab 713 is selected, with a window 721 being displayed representing a complete shoulder replacement joint, which it will be appreciated is generated upon completion of the following planning phase.

At step 615, key features within the 3D models can be identified. This can be performed automatically by having the server 210 and/or client device 220 analyse the shape of the anatomical models, in this case the models of the humerus or scapula, or manually by having the user select key points on the models using a mouse or other input device. This could also be performed using a combination of automatic and manual processes, for example by having approximate locations of key features identified automatically and then having these refined manually if required.

Examples of this process are shown in FIGS. 7C and 7E for the scapula and humerus respectively. In each case, the key points tab 712 is selected so that the user interface 700 displays the relevant model in the window 721, and includes inputs in the left side bar 730 allowing each of the key features to be selected. In the example of FIG. 7C, the right side bar 740 shows a fit model used to identify the glenoid centre, with this allowing the user to select different fit models as required. Additionally, in the example of FIG. 7F, the humerus tab 715 is selected allowing a user to define a feature in the form of a desired cut-plane for the cutting of the humerus, to allow for attachment of an implant, such as a socket. In this instance, the left side bar 730 includes controls allowing the position, including the location and angle of the cutting plane, to be adjusted.

The example interface of FIGS. 7C and 7E is displayed on a display screen in two dimensions, but it will be appreciated that digital reality representations, such as virtual reality representation, could also be used to allow the model to be viewed in three dimensions. An example of this is shown in FIG. 7G. In this example, an interface 750 is displayed in the form of a virtual reality environment, with a model 760 of the scapula including identified key points 761 displayed therein. In this instance, a representation of a hand is displayed, corresponding to a position and orientation of a controller, allowing a user to manipulate the model and view the model from different viewpoints.

At step 620, the user selects one or more components, such as implants, tools or guides to be used in the procedure, with corresponding models being retrieved. This is typically achieved by retrieving pre-defined model data associated with the implants and tools provided by a supplier, with the respective model data being retrieved from the server 210 as needed.

At step 625, a visualisation including the component can then be displayed on the user interface, allowing the user to align the component as needed at step 630. Again, this can be performed automatically, for example by positioning the component based on the identified key features, and/or manually, based on visual inspection of the model and user input commands.

An example of this process is shown in FIG. 7D. In this case, the glenoid tab 714 is selected so that the user interface 700 displays the scapula model in the window 721, including the implant attached to the glenoid of the scapula. A representation of the position of the implant 723.1, 724.1 is also shown overlaid on the scan data in the windows 723, 724, whilst the left side bar 730 shows a representation of the implant, together with controls allowing the position of the implant to be adjusted.

Again it will be appreciated that this process could also be performed using a digital reality representation and an equivalent virtual reality visualisation is shown in FIG. 7H. In this instance, again a model of the scapula 760 is shown, together with an attached implement 762. A menu 780 is displayed allowing the user to control the visualisation, with a second menu 790 being provided including control inputs to allow a position of the implant relative to the glenoid to be controlled.

Once alignment of an implant or surgical tool has been determined, the operative position of the guide needed to achieve the alignment can be calculated at step 635. This is typically performed automatically by the client device 220 and/or server 210, simply by positioning the guide relative to the humerus or glenoid in such a manner that alignment of the surgical tool or implant is achieved. It will be appreciated however that this is stage might not be required if the guide itself was positioned during steps 625 and 630.

Once the operative position of the guide has been determined, a custom guide shape can be generated at step 640, by the client device 220 and/or server 210. Typically this involves calculating the shape of the guide, so that the guide shape conforms to a shape of an outer surface of the anatomical part when the guide is in the operative position. This could be achieved in any appropriate manner, but will typically involve using a template shape, and then subtracting from the template, any overlap between the template shape and the anatomy.

At step 645, guide markings can be generated. The guide markings are typically fiduciary markings or similar that are to be displayed on the guide, surgical tools or patient, allow a position of the guide to be detected using sensors, such as an imaging device. In one example, fiducial markings, such as infrared navigation markers, QR codes, or April Tags, described in “AprilTag: A robust and flexible visual fiducial system” by Edwin Olson in Proceedings of the IEEE International Conference on Robotics and Automation (ICRA), 2011, are used, which allow a physical location of the guide to be derived through a visual analysis of the fiducial markers in the captured images.

Once the guide shape and any required markings have been generated, guide data can be generated by the client device 220 or server 210 at step 650. Typically this involves generating data that can be used in an additive and/or subtractive manufacturing process, and in one particular example, in a 3D printing process, such as an STL file or equivalent. The guide data can then be provided to a manufacturer, or an STL file can be sent directly to a printer, allowing the custom surgical guide to be manufactured at step 655. Once manufactured, any required markings can be added, for example by printing the markings thereon.

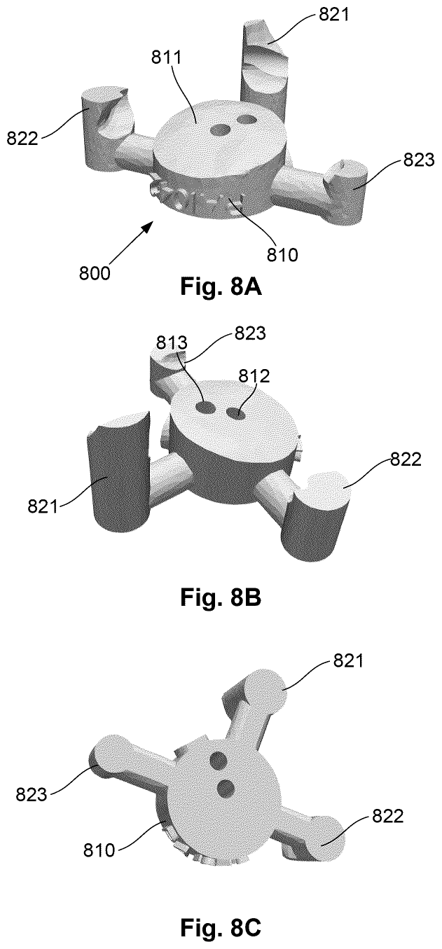

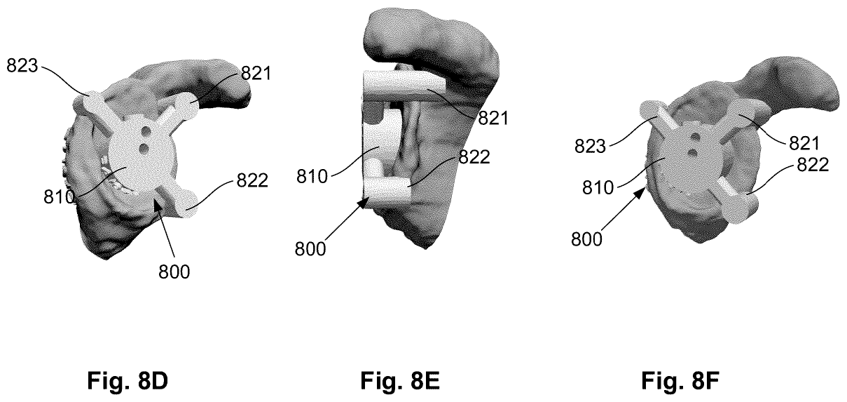

An example of a custom glenoid guide for use in a shoulder replacement procedure will now be described with reference to FIGS. 8A to 8F.

In this example, the glenoid guide 800 includes a generally cylindrical glenoid guide body 810 including an underside 811 configured to abut the glenoid in use. The body 810 includes a central hole 812 that receives a K-wire for guiding positioning of the implant, and a superior hole 813 in which a K-wire is temporarily inserted to create a mark used as an indicator, so that rotation of the glenoid implant can be controlled during insertion. An anterior hole (not shown) is also provided, which can receive a surgical tool used to aid in placement and stability of the guide.

The body 810 includes an anterosuperior arm 821 that sits and articulates inferior to the coracoid process, and extends across the glenoid vault and over the bony rim of the glenoid in use, an anteroinferior arm 822 that sits along the anteroinferior aspect of the glenoid and glenoid vault, and extends over the bony rim of the glenoid and a posterosuperior arm 823 that sits on the bony glenoid rim.

The combination of the arms 821, 822, 823 and shaped underside 811 of the body 810 ensures that the guide can only sit in one position on the glenoid, thereby ensuring the K-wires and markings are correctly positioned, so that the implant is in turn attached to the glenoid in a desired position, orientation and rotation, as shown in FIGS. 8D to 8F.

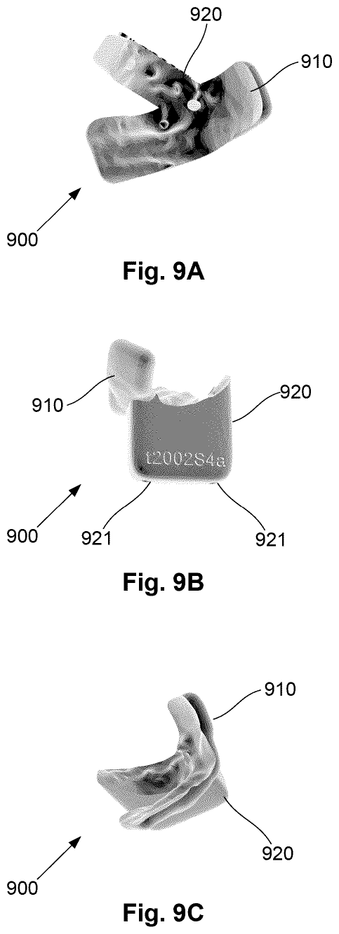

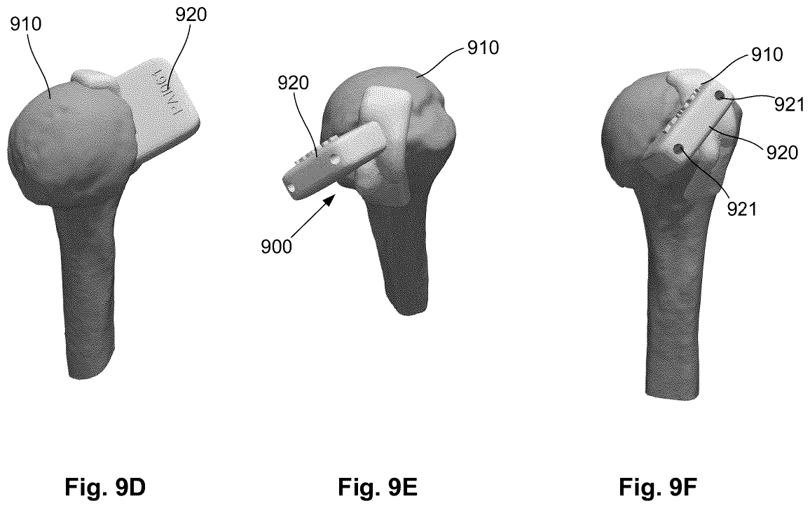

An example of a custom humeral guide for use in a shoulder replacement procedure will now be described with reference to FIGS. 9A to 9F.

In this example, the humeral guide 900 includes a humeral guide body 910 that attaches to the humeral head, extending from an articular surface of a humeral head down the bicipital groove of the humerus, and a humeral guide arm 920 configured to extend from the body and including one or more holes 921 configured to receive surgical pins to allow for attachment of a cutting block to the humerus. In this example, an underside of the humeral guide body is shaped to conform to a profile of the humeral head, allowing the humeral guide to be attached at a fixed position and orientation relative to the humeral head. This ensures surgical pins are inserted into the humeral head at a desired location, in turn ensuring cutting of the humeral head is performed as required.

In addition to allowing the above described system to be used to design a custom guide, the system can be used to allow a surgical plan for the procedure to be developed, and then displayed using a mixed or augmented reality display, so that the steps in the surgical procedure can be displayed superimposed on the real world. This allows intraoperative decision making and allows the surgeon to have access to pertinent information during the procedure, and an example of this process will now be described.

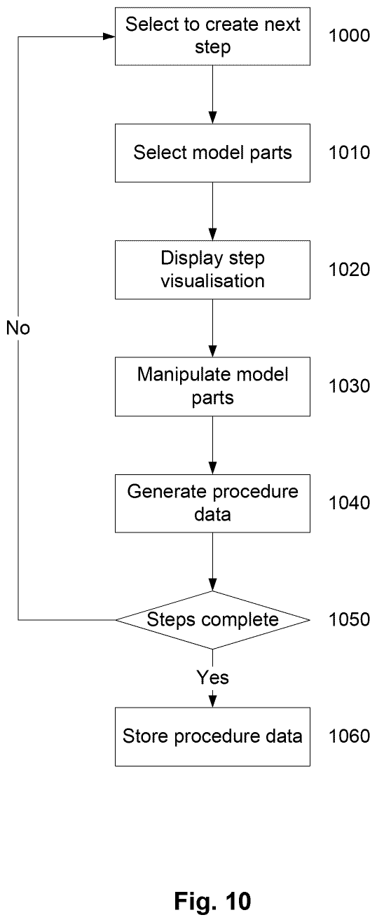

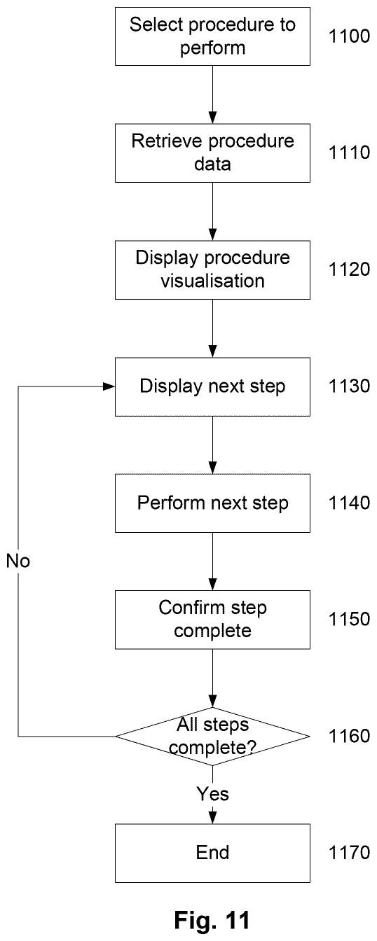

An example of the process for planning a surgical procedure will now be described with reference to FIG. 10.

In this example, at step 1000 the user uses an interface similar to the interfaces described above with respect to FIGS. 7A to 7H to create a next step in the surgical procedure.

At step 1010, the user selects one or more model parts, such as the anatomical part, and one or more components, such as a surgical tool, surgical guide or implant, used in performing the step. A visualisation of the respective model parts is then displayed by the client device 220, at step 1020, allowing the user to manipulate the model parts to represent the respective step at step 1030. For example, an initial step might simply involve the placement of a respective guide on the humerus or glenoid respectively, in which case the user can manipulate a visualisation including models of the guide and anatomical part, until the guide is in position. The user can then indicate the step is complete, allowing the client device to generate procedure data for the step at step 1040.

It will be appreciated that the above example would effectively represent a static image of a completed step, but movement information could be recorded, showing the movements required to position the guide, allowing an animation of how a step is performed to be generated.

Once a step is finished, it is determined if all steps are completed, typically based on user input at step 1050. If further steps are required the process to return to step 1000, enabling further steps to be defined, otherwise procedure data indicative of the steps is stored by the client device 220 and/or server 210 at step 1060.

In addition to defining steps performed in the procedure, the procedure data can include any other information relevant to, or that could assist with, performing the surgical procedure. Such information could include, but is not limited to scan data indicative of scans performed on the subject, subject details including details of the subject's medical records, symptoms, referral information, or the like, information or instructions from an implant manufacturer, or the like.

Accordingly, it will be appreciated that this allows a user to develop a sequence of steps representing the surgical procedure to be performed, allowing these, together with other additional information to be displayed to a user during the surgical phase. An example of this will now be described with reference to FIG. 11.

In this example, at step 1100 a procedure to be performed is selected, typically by having the user select a particular patient via a user interface provided in a display device 230. Procedure data is then retrieved by the server 210 and/or client device 220 at step 1110, allowing a procedure visualisation to be generated and displayed on the display device 230 at step 1120.

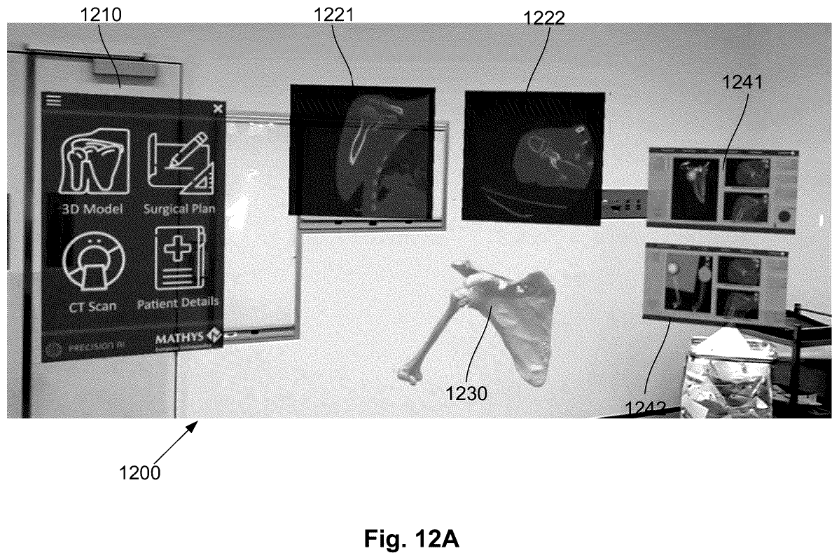

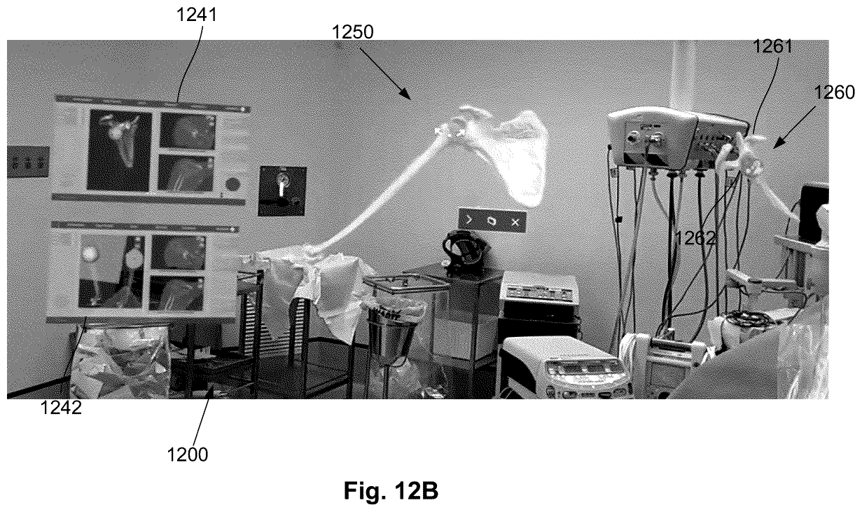

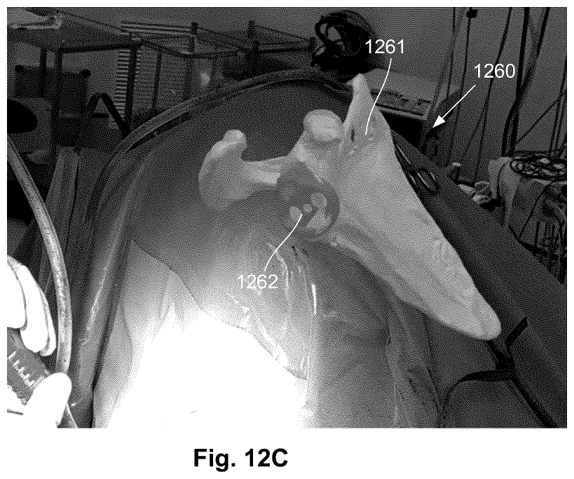

An example procedure visualisation displayed using an augmented reality display will now be described with reference to FIGS. 12A to 12C.

In this example, the visualisation includes a user interface 1200, including a menu 1210, allowing the user to select the particular information that is displayed, such as 3D models, the surgical plan, CT scans, or patient details. In this example, the procedure visualisation further includes scan representations, including coronal and sagittal CT scans 1221, 122, and the resulting anatomical model 1230 derived from the scans, which in this example include the scapula and humerus. It will be appreciated that these visual elements can be dynamic, allowing the user to manipulate the model and view this from different viewpoints, and/or view different ones of the scans.

Images 1241, 1242 of the user interface used in the planning process are also shown, allowing the user to review particular steps in the planning procedure, with a model 1250 of the resulting implant also being displayed. Additionally, a step model 1260 of a respective step in the procedure is shown, in this example including the scapula 1261 and implant 1262, allowing the user to view how the implant should be attached.

In this example, a next step can be displayed at 1130, allowing the user to perform the step at step 1140, and visually compare the results with the intended outcome displayed in the model 1260. Assuming the step is completed to the user's satisfaction, this can be indicated via suitable input at step 1150. It is then determined by the client device 220 and/or server 210 if all steps are complete at step 1160, and if not the process returns to step 1130 allowing further steps to be displayed by updating the model 1260 and optionally the user interface screens 1241, 1242, otherwise the process ends at step 1170.

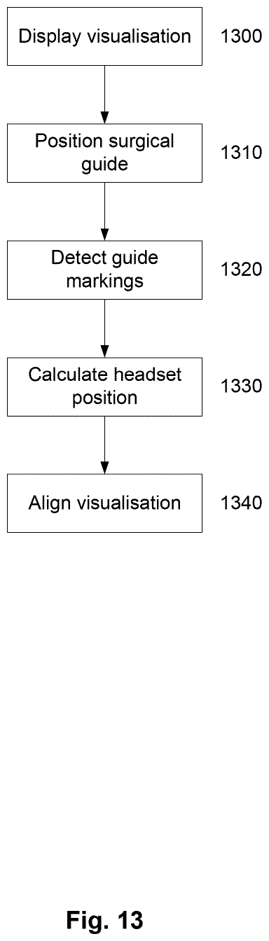

During the above described process, the model 1260 can be displayed aligned with the subject anatomy, to thereby further assist in performing the procedure. An example of this process will now be described with reference to FIG. 13.

In this example, at step 1300, a visualisation including the model 1260 is displayed to the user via the display device 230, for example as part of the above described process.

At step 1310, the surgical guide is positioned. This could include attaching the guide to the subject's anatomy, for example attaching the glenoid guide to the glenoid, or could simply include holding the guide so that it is visible to a sensor, such as an imaging device on the display device 230. The markings are detected by the client device 220 within images captured by the imaging device at step 1320, allowing a headset position relative to the markings to be calculated at step 1330. The client device 220 can then update the visualisation so that this is displayed with a guide in the model 1260 aligned with the actual guide at step 1340.

In the event that the guide is attached to the subject, this will align the subject's anatomy with the model 1260 so that the model is overlaid on and aligned with the subject. This in turn can help the user compare the placement of the implant and/or tools in subsequent steps, to ensure these are positioned as intended.

Accordingly, the above described system and process enables a surgical procedure to be planned and implemented more effectively. In particular, this can be used to generate a series of models, which in turn act to guide a user such as a surgeon, in carrying out the required steps to perform a procedure, allowing visual comparison to be used to ensure the procedure is performed correctly. This can advantageously be performed using augmented or mixed reality, enabling the surgeon to more easily view relevant information without this preventing the surgeon performing the procedure.

To prove accuracy of the surgical guides and hence the planning approach a cadaveric study was completed on Jul. 12, 2020.

This study involved the evaluation of a total of 18 glenoid and 18 humeral guides. Each guide was produced from a distinct surgical plan and preoperative CT from the specific donor. For each of the final planned positions of the prosthesis, one custom patient-specific glenoid guide and one humeral guide was constructed, and 3D printed in biocompatible nylon (PA12). These guides were then used intraoperatively to assist with the drilling and placement of the glenoid k-wires and humeral head studs.

Once inserted a post-operative CT scan was acquired using the identical protocol as in the preoperative CTs. This procedure was subsequently repeated of a total of three times for each glenoid and humerus. No prostheses were inserted, the subsequent analysis of the accuracy of the PSIs were based on the planned vs measured placement of the k-wires/studs as the presence of metal objects in the CT scan field can lead to severe streaking artifacts which would reduce the accuracy of any true post-operative measurements.

The study was conducted at the Medical Engineering Research Facility (MERF), Institute of Health and Biomedical Innovation (IHBI), Queensland University of Technology, Staib Rd, Chermside, QLD 4032. Ethics approval was provided by the University of Queensland (#2019003068) and can be provided on request. Three surgeons were involved in the study, Drs Benjamin Kenny, Ali Kalhor and Praveen Vijaysegaran, all with subspecialty post fellowship qualifications in shoulder surgery.

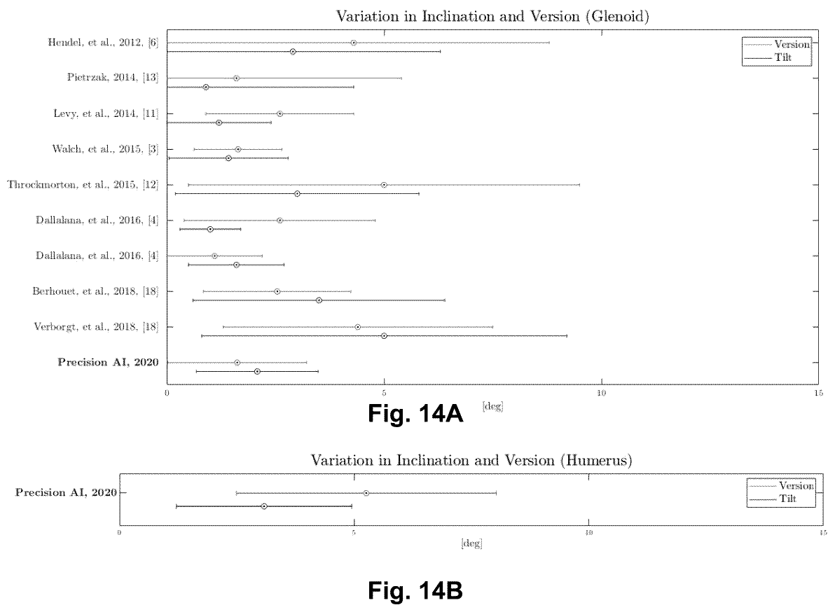

Results are shown in Tables 1 and 2 and FIGS. 14A and 14B respectively for the glenoid and humeral guides. These results demonstrate that the guides and planning approach work effectively, and lead to improved outcomes.

TABLE 1

| | | | | | Variation in | Variation in |
|---|---|---|---|---|---|---|
| | | | | | Inclination | retroversion |
| | | | | | (*) | (*) |
| | | | | | Average, | Average |
| | | Cadaveric | | | standard | standard |
| Author and | | or in vivo | | Measure | deviation, | deviation, |
| Year | N | study | TSA | Reference | range | range |
| Hendel, et al., | 15 | In vivo | Anatomic | Implant | 2.9 ± 3.4 | 4.3 ± 4.5 |
| 2012 [6] | | | | | (−13.0-6.0) | (−8.0-12.0) |
| Walch, et al., | 18 | Cadaveric | Anatomic | Pin | 1.42 ± 1.37 | 1.64 ± 1.01 |
| 2015, [3] | | | | | (0.09-4.55) | (0.17-3.21) |
| Throckmorton, | 18 | Cadaveric | Anatomic | Implant | 3.0 ± 4.3 | 5.0 ± 4.8 |
| et al., 2015, [12] | | | | | (no range | (no range |
| | | | | | reported) | reported) |
| Dallalana, et al., | 10 | In vivo | Anatomic | Implant | 1.0 ± 0.7 | 2.6 ± 2.2 |
| 2016, [4] | | | | | (0.3-2.7) | (0.2-7.3) |
| Berhouet, el al., | 10 | In vivo | Anatomic | Implant | 3.5 ± 2.9 | 2.5 ± 1.7 |
| 2018, [17] | | | | | (no range | (no range |
| | | | | | reported) | reported) |
| Levy, et al., | 14 | Cadaveric | Reverse | Pin | 1.2 ± 1.2 | 2.6 ± 1.7 |
| 2014. [11] | | | | | (0.1-4.7) | (0.1-8.4) |
| Throckmorton, | 17 | Cadaveric | Reverse | Implant | 4.0 ± 4.6 | 6.0 ± 7.0 |
| et al., 2015, [12] | | | | | (no range | (no range |
| | | | | | reported) | reported) |
| Dallalana, et al., | 10 | In vivo | Reverse | Implant | 1.6 ± 1.1 | 1.1 ± 1.1 |
| 2016, [4] | | | | | (0.2-4.5) | (0.1-4.0) |
| Verborgt, et al., | 32 | In vivo | Reverse | Implant | 5.0 ± 4.2 | 4.4 ± 3.1 |
| 2018, [18] | | | | | (0.1-14.5) | (0.3-13.7) |
| Pietrzak, 2014, | 12 | Cadaveric | Both | Implant | 0.9 ± 3.4 | 1.6 ± 3.8 |
| [13] | | | | Pin | (no range | (no range |
| | | | | | reported) | reported) |
| Precision Al, | 18 | Cadaveric | Both | Pin | 2.08 ± 1.4 | 1.62 ± 1.6 |
| 2020 | | | | | (0.1-4.2) | (0.1-5.7) |

TABLE 2

| | | | | | Variation | |
|---|---|---|---|---|---|---|
| | | | | | in | Variation in |
| | | | | | Inclination | retroversion |
| | | | | | (*) | (*) |
| | | | | | Average, | Average |
| | | | | | standard | standard |
| Author and | | Cadaveric or | | Measure | deviation, | deviation, |
| Year | N | in vivo study | TSA | Reference | range | range |
| Precision Al, | 18 | Cadaveric | Both | Studs | 3.08 ± 1.87 | 5.26 ± 2.77 |
| 2020 | | | | | (0.4-6.9) | (1.3-8.9) |

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) A surgical system for use in performing a surgical implant procedure on a biological subject, the system including:
a) in a planning phase:
i) a planning display device;
ii) one or more planning processing devices configured to:
(1) acquire scan data indicative of a scan of an anatomical part of the subject;
(2) generate model data indicative of:
(a) an anatomical part model generated using the scan data; and,
(b) at least one of:
(i) a surgical guide model representing a surgical guide used in positioning a surgical implant;
(ii) an implant model representing the surgical implant; and,
(iii) a tool model representing the surgical tool used in performing the surgical procedure;
(3) cause a planning visualisation to be displayed to a user using the planning display device, the planning visualisation being generated at least in part using the model data; and,
(4) manipulate the planning visualisation in accordance with user input commands indicative of interaction with the planning visualisation to at least one of:
(a) calculate a custom guide shape for the surgical guide; and,
(b) at least partially plan the surgical procedure; and,
b) in a surgical phase:
i) a surgical guide configured to assist in aligning an implant with the anatomical part in use;
ii) a procedure display device; and,
iii) one or more procedure processing devices configured to cause a procedure visualisation to be displayed to a user using the procedure display device, the procedure visualisation being generated at least in part using the model data and being displayed whilst the surgical procedure is performed.

2) A system according to claim 1, wherein the one or more planning processing devices use manipulation of the planning visualisation to:
a) determine an operative position of the surgical guide relative to the anatomical part; and,
b) calculate a custom guide shape for the surgical guide based on the operative position.

3) A system according to claim 1 or claim 2, wherein the one or more planning processing devices are configured to use user input commands to determine an alignment indicative of a desired relative position of the anatomical part model and at least one of:
a) the surgical implant; and,
b) a surgical tool.

4) A system according to claim 3, wherein the one or more planning processing devices are configured to determine an operative position of the surgical guide relative to the anatomical part at least in part using the alignment.

5) A system according to claim 3 or claim 4, wherein the one or more planning processing devices are configured to determine the alignment at least in part by having a user at least one of:
a) identify key anatomical features in the representation of the anatomical part model, the alignment being determined based on the key anatomical features; and,
b) position the surgical implant relative to the anatomical part in the visualisation.

6) A system according to any one of the claims 3 to 5, wherein the planning visualisation includes one or more input controls allowing a user to adjust the alignment.

7) A system according to any one of the claims 1 to 6, wherein the one or more planning processing devices generate procedure data indicative of a sequence of steps representing progression of the surgical implant procedure.

8) A system according to any one of the claims 1 to 7, wherein the one or more planning processing devices generate the procedure data at least in part by:
a) causing the planning visualisation to be displayed;
b) using user input commands representing user interaction with the planning visualisation to create each step, each step being indicative of a location and/or movement of at least one of:
i) a surgical tool;
ii) a surgical guide; and,
iii) a surgical implant; and,
c) generate the procedure data using the created steps.

9) A system according to claim 8, wherein the one or more procedure processing devices are configured to use the procedure data to cause the procedure visualisation to be displayed.

10) A system according to claim 8 or claim 9, wherein the one or more procedure processing devices are configured to:
a) determine when a step is complete in accordance with user input commands; and,
b) cause the procedure visualisation to be updated to display a next step.

11) A system according to any one of the claims 1 to 10, wherein the procedure visualisation is indicative of at least one of:
a) the scan data;
b) the anatomical part model;
c) a model implant; and,
d) one or more steps.

12) A system according to any one of the claims 1 to 11, wherein the one or more procedure processing devices are configured to:
a) determine a procedure display device location with respect to:
i) the surgical guide; or
ii) the anatomical part of the subject; and,
b) cause the procedure visualisation to be displayed in accordance with the procedure display device location so that:
i) a visualisation of the surgical guide model is displayed overlaid on the surgical guide; or
ii) a visualisation of the anatomical part model is displayed overlaid on the anatomical part of the subject.

13) A system according to claim 12, wherein the one or more procedure processing devices are configured to determine the procedure display device location by at least one of:
a) using signals from one or more sensors;
b) using user input commands;
c) performing image recognition on captured images; and,
d) detecting coded data present on at least one of the surgical guide, surgical tools and the subject.

14) A system according to claim 13, wherein the captured images are captured using an imaging device associated with the procedure display device.

15) A system according to any one of the claims 1 to 14, wherein the planning or procedure visualisation includes a digital reality visualisation, and wherein the one or more processing devices are configured to allow a user to manipulate visualisation by interacting with at least one of:
a) the anatomical part;
b) the surgical implant;
c) a surgical tool; and,
d) to surgical guide.

16) A system according to any one of the claims 1 to 15, wherein at least one of the planning and procedure display devices is at least one of:
a) an augmented reality display device; and,
b) a wearable display device.

17) A system according to any one of the claims 1 to 16, wherein the surgical implant includes at least one of:
a) a prosthesis;
b) an orthopaedic shoulder prosthesis;
c) a ball and socket joint;
d) a humeral implant attached to a humeral head of the subject;
e) a glenoidal implant attached to a glenoid of the subject;
f) ball attached via a stem to the humeral head or glenoid of the subject; and,
g) a socket attached using a binding material to the glenoid or humeral head of the subject.

18) A system according to any one of the claims 1 to 17, wherein the surgical guide includes a glenoidal guide for attachment to a glenoid of the subject, and wherein the glenoidal guide includes:
a) a glenoidal guide body configured to abut the glenoid in use, the glenoidal guide body including one or more holes for use in guiding attachment of an implant to the glenoid; and,
b) a number of glenoidal guide arms configured to engage an outer edge of the glenoid to secure the glenoidal guide in an operative position.

19) A system according to claim 18, wherein an underside of the glenoid body is shaped to conform to a profile of the glenoid.

20) A system according to claim 18 or claim 19, wherein the one or more holes include:
a) a central hole configured to receive a K-wire for guiding positioning of the implant;
b) a superior hole for configured to receive a temporary K-wire used to act as an indicator of rotation and placement of the glenoid implant during insertion; and,
c) an anterior hole configured to receive a surgical tool used to aid in placement and stability of the guide.

21) A system according to any one of the claims 18 to 20, wherein the glenoidal guide arms include:
a) an anterosuperior arm configured to sit and articulate inferior to the coracoid process, and extend across the glenoid vault and over the bony rim of the glenoid in use;
b) an anteroinferior arm configured to sit along the anteroinferior aspect of the glenoid and glenoid vault and extend over the bony rim of the glenoid; and,
c) a posterosuperior arm configured to sit on the bony glenoid rim.

22) A system according to any one of the claims 1 to 21, wherein the surgical guide includes a humeral guide for attachment to a humerus of the subject, and wherein the humeral guide includes:
a) a humeral guide body configured to extend from an articular surface of a humeral head down the bicipital groove of the humerus; and,
b) a humeral guide arm configured to extend from the body and including one or more holes configured to receive surgical pins to allow for attachment of a cutting block to the humerus.

23) A system according to claim 22, wherein an underside of the humeral guide body is shaped to conform to a profile of the humeral head.

24) A method for performing a surgical implant procedure on a biological subject, the method including:
a) in a planning phase using one or more planning processing devices to:
i) acquire scan data indicative of a scan of an anatomical part of the subject;
(1) generate model data indicative of:
(a) an anatomical part model generated using the scan data; and,
(b) at least one of:
(i) a surgical guide model representing a surgical guide used in positioning a surgical implant;
(ii) an implant model representing the surgical implant; and,
(iii) a tool model representing the surgical tool used in performing the surgical procedure;
(2) cause a planning visualisation to be displayed to a user using the planning display device, the planning visualisation being generated at least in part using the model data; and,
(3) manipulate the planning visualisation in accordance with user input commands indicative of interaction with the planning visualisation to at least one of:
(a) calculate a custom guide shape for the surgical guide; and,
(b) at least partially plan the surgical procedure; and,
b) in a surgical phase:
i) using a surgical guide to assist in aligning an implant with the anatomical part in use; and,
ii) using one or more procedure processing devices to display a procedure visualisation to a user using a procedure display device, the procedure visualisation being generated at least in part using the model data and being displayed whilst the surgical procedure is performed.

25) A surgical system for planning a surgical implant procedure on a biological subject, the system including:
a) a planning display device;
b) one or more planning processing devices configured to:
i) acquire scan data indicative of a scan of an anatomical part of the subject;
(1) generate model data indicative of:
(a) an anatomical part model generated using the scan data; and,
(b) at least one of:
(i) a surgical guide model representing a surgical guide used in positioning a surgical implant;
(ii) an implant model representing the surgical implant; and,
(iii) a tool model representing the surgical tool used in performing the surgical procedure;
(2) cause a planning visualisation to be displayed to a user using the planning display device, the planning visualisation being generated at least in part using the model data; and,
(3) manipulate the planning visualisation in accordance with user input commands indicative of interaction with the planning visualisation to at least one of:
(a) calculate a custom guide shape for the surgical guide; and,
(b) at least partially plan the surgical procedure.

26) A surgical system for performing a surgical implant procedure on a biological subject, the system including:
a) a surgical guide configured to assist in aligning an implant with the anatomical part in use;
b) a procedure display device; and,
c) one or more procedure processing devices configured to cause a procedure visualisation to be displayed to a user using the procedure display device, the procedure visualisation being generated at least in part using model data and being displayed whilst the surgical procedure is performed.

27) A method for planning a surgical implant procedure on a biological subject, the method including using one or more planning processing devices to:
a) acquire scan data indicative of a scan of an anatomical part of the subject;
b) generate model data indicative of:
i) an anatomical part model generated using the scan data; and,
ii) at least one of:
(1) a surgical guide model representing a surgical guide used in positioning a surgical implant;
(2) an implant model representing the surgical implant; and,
(3) a tool model representing the surgical tool used in performing the surgical procedure;
c) cause a planning visualisation to be displayed to a user using the planning display device, the planning visualisation being generated at least in part using the model data; and,
d) manipulate the planning visualisation in accordance with user input commands indicative of interaction with the planning visualisation to at least one of:
i) calculate a custom guide shape for the surgical guide; and,
ii) at least partially plan the surgical procedure.

28) A method for performing a surgical implant procedure on a biological subject, the method including:
a) using a surgical guide generated using a to assist in aligning an implant with the anatomical part in use; and,
b) using one or more procedure processing devices to display a procedure visualisation to a user using a procedure display device, the procedure visualisation being generated at least in part using model data and being displayed whilst the surgical procedure is performed.

29) A humeral guide for a shoulder prosthesis implant procedure, the humeral guide being for attachment to a humerus of the subject, and including:
a) a humeral guide body configured to extend from an articular surface of a humeral head down the bicipital groove of the humerous; and,
b) a humeral guide arm configured to extend from the body and including one or more holes configured to receive surgical pins to allow for attachment of a cutting block to the humerous.

30) A humeral guide according to claim 29, wherein an underside of the humeral guide body is shaped to conform to a profile of the humeral head.
